(19) United States
(12) Patent Application Publication
(10) Pub. No.: US 2026/0283511 A1
(43) Pub. Date: Sep. 24, 2026

(54) MONITORING DEVICE

(71) Applicant: Murata Vios, Inc., Woodbury, MN (US)

(72) Inventor: Yuzo HIGASHIYAMA, Woodbury, MN (US)

(21) Appl. No.: 19/566,158

(22) Filed: Mar. 13, 2026

Related U.S. Application Data
(60) Provisional application No. 63/774,371, filed on Mar. 19, 2025.

(51) Int. Cl. A61B 5/1455; A61B 5/00; A61B 90/96

(52) U.S. Cl. CPC A61B 5/14552; A61B 5/6826; A61B 90/96; A61B 2560/0214; A61B 2560/0462

(57) ABSTRACT

A monitoring device includes a transparent layer and a data representation located underneath the transparent layer such that the data representation is visible through the transparent layer. The monitoring device is configured to monitor a user when the monitoring device is attached to the user.

### CROSS REFERENCE TO RELATED APPLICATIONS

This application claims the benefit of U.S. Patent Application No. 63/774,371 filed on Mar. 19, 2025. The entire contents of this application are hereby incorporated by reference.

### BACKGROUND OF THE INVENTION

### 1. Field of the Invention

The present invention relates to monitoring devices. More specifically, the present invention relates to monitoring devices that include a data representation, such as a printed bar code or text.

### 2. Description of the Related Art

Medical devices that are designed to be worn on a user's body must undergo thorough cleaning, including disinfection, both before and after use to prevent infection. But repeated cleaning can result in the removal or degradation of critical information printed on the medical device's surface. For instance, medical devices are often required to display a Unique Device Identifier (UDI) for traceability purposes, and this information may become illegible over time due to wear from cleaning processes.

Additionally, wearable medical devices intended for prolonged use, such as SpO2 sensors, must employ biocompatible materials to ensure safety. This requirement restricts the range of materials suitable for printing and increases costs associated with manufacturing.

Efforts to address these challenges have included the use of durable and biocompatible inks. Laser printing has also been explored as an alternative because laser printing eliminates the need for ink and avoids issues related to biocompatibility. However, laser printing on certain materials, such as rubber, continues to face durability concerns, leaving room for improvement in this area.

### SUMMARY OF THE INVENTION

To overcome the problems described above, example embodiments of the present invention provide monitoring devices that can be attached to users and that each include a data representation, such as a printed barcode or text, underneath a transparent layer that directly contacts the user when the monitoring devices are attached to the user. The monitoring devices can be monitoring devices that can be attached to fingers of users, including, for example, a pulse oximeter. But other monitoring devices that are attached to a patient can also be used.

According to an example embodiment of the present invention, a monitoring device includes a transparent layer and a data representation located underneath the transparent layer such that the data representation is visible through the transparent layer. The monitoring device is configured to monitor a user when the monitoring device is attached to the user.

The data representation can include a Unique Device Identifier of the monitoring device. The data representation can include a machine-readable barcode. The machine-readable barcode can include a one-dimensional barcode and/or a two-dimensional barcode. The data representation can include human-readable information. The human-readable information can be text and/or alphanumeric characters. The data representation can include a printed data representation.

The transparent layer can include rubber. The monitoring device can have a C-shape, and the transparent layer can be on an inner surface of the monitoring device. The monitoring device can be configured such that the transparent layer is in direct contact with the user when the monitoring device is attached to the user. The monitoring device can include a digit attacher configured to be attached to a finger of the user such that the transparent layer is in direct contact with the finger of the user when the monitoring device is attached to the finger of the user.

According to an example embodiment of the present invention, a monitoring device system includes the monitoring device of one of the various example embodiments of the present invention attached to the user.

The monitoring device can be attached to a finger of the user. The transparent layer can be in direct contact with the finger of the user. The monitoring device system can further include a power supply connected to the monitoring device and a measurement device connected to the monitoring device.

According to an example embodiment of the present invention, a monitoring device includes a substrate having a C-shape configured so that the monitoring device can be attached to a user's digit, a transparent layer that is on an inner surface of the substrate and that directly contacts the user's digit when the monitoring device is attached to the user's digit, and a data representation located under the transparent layer such that the data representation can be viewed through the transparent layer.

The data representation can include a Unique Device Identifier. The data representation can include a machine-readable barcode. The machine-readable barcode can include a one-dimensional barcode and/or a two-dimensional barcode. The data representation can include human-readable information. The human-readable information can be text and/or alphanumeric characters. The data representation can include a printed data representation. The transparent layer can include rubber.

According to an example embodiment of the present invention, a monitoring device system includes the monitoring device of one of the various example embodiments of the present invention, a power supply connected to the monitoring device, and a measurement device connected to the monitoring device.

The monitoring device can be attached to the user's digit.

The above and other features, elements, characteristics, steps, and advantages of the present invention will become more apparent from the following detailed description of example embodiments of the present invention with reference to the attached drawings.

### BRIEF DESCRIPTION OF THE DRAWINGS

FIG. 1 shows a perspective view of a pulse oximeter that can wrap around the side of a user's finger.

FIG. 2 shows a cross-sectional view of the pulse oximeter of FIG. 1.

FIG. 3 shows the pulse oximeter of FIG. 1 wrapped around the side of a user's finger.

FIG. 4 shows a pulse oximeter that wraps around the tip of a user's finger.

FIG. 5 shows the location of a data representation on the pulse oximeter of FIG. 3.

### DETAILED DESCRIPTION OF EXAMPLE EMBODIMENTS

A monitoring device can be attached to a user to monitor the user. For example, the user can be a patient in a doctor's office or in a hospital. The monitoring device can include a digit attacher that allows the monitoring device to be attached to a user's digit, e.g., a finger or a toe. But it is also possible to attach the monitoring device to another portion of the user. The digit attacher can include a C-shaped substrate that allows the monitoring device to be attached to the user's digit. The monitoring device can include a transparent layer on an inner surface of the substrate. If the monitoring device includes, for example, a pulse oximeter, then the transparent layer can transmit both visible light and infrared light.

The monitoring device can include a data representation that includes information related to the monitoring device. The data representation can include, for example, a Unique Device Identifier (UDI) that uniquely identifies the monitoring device. The UDI can be used to track the monitoring device. In some applications, the UDI can be associated with a specific user so that the information gathered by the monitoring device, including, for example, the specific user's vital sign data, can be associated with the specific user. For example, the UDI can be used in remote monitoring to ensure that the monitored data is shown for the correct user.

The data representation can include a machine-readable barcode and/or human-readable information. The machine-readable barcode can include a one-dimensional barcode and/or can include a two-dimension barcode, such as a quick-response (QR) code. The human-readable information can include text and/or alphanumeric characters.

The data representation can be located on an inner portion of the monitoring device and can be covered by the transparent layer so that the data representation is visible through the transparent layer. The data representation can be printed on the monitoring device. The substrate of the monitoring device can provide a flat or substantially flat surface on which the data representation can be printed. The transparent layer can make direct contact with a user (e.g., direct contact with the user's skin) and can include, for example, a biocompatible transparent rubber or other suitable material. The monitoring device should be cleaned before and/or after each use. The transparent layer can protect the data representation during surface cleaning because the data because the data representation is not on the surface of the device, which increases the durability of the monitoring device. By including the data representation underneath the transparent layer, the material used for the data representation does not have to be biocompatible because the data representation is not in direct contact with the user. Because the data representation does not have to use a biocompatible material and because the data representation is protected by the transparent layer, the cost of the monitoring device can be decreased, and the durability of the monitoring device can be increased.

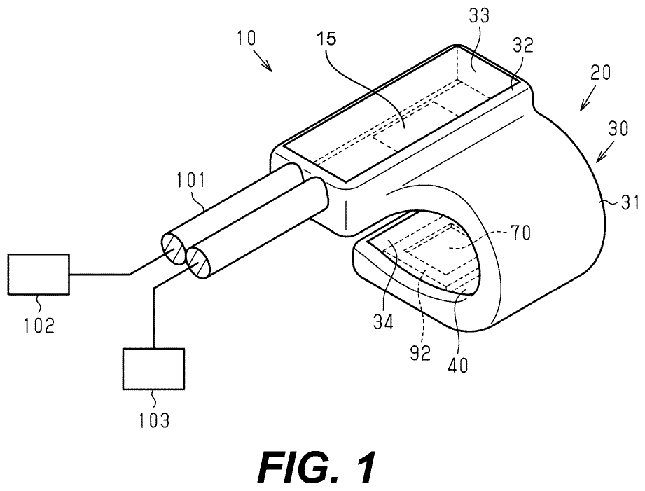

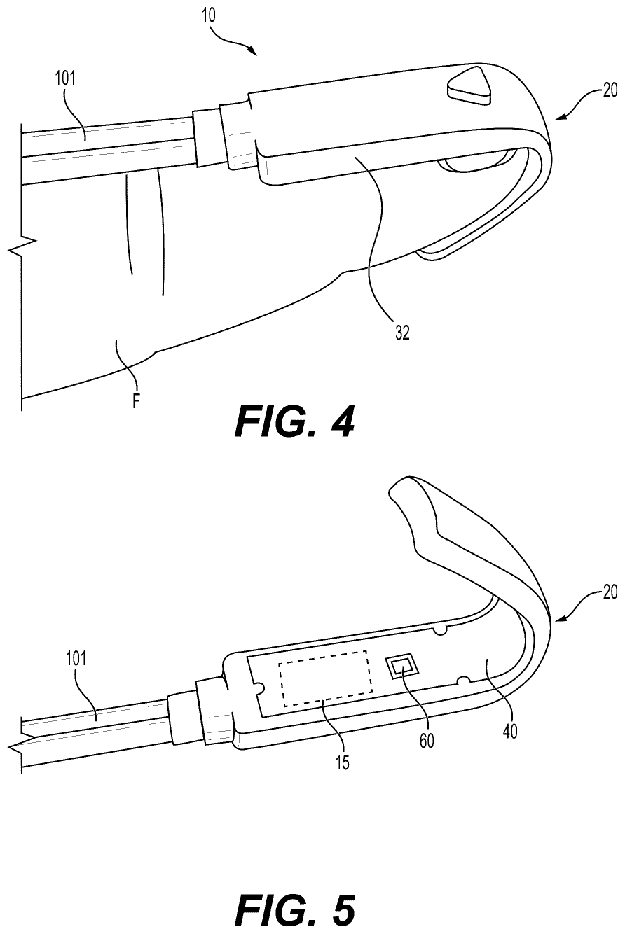

The monitoring devices can be the pulse oximeters 10 shown in FIGS. 1-5 or can be other suitable medical devices. FIGS. 1-5 show pulse oximeters 10 that can be wrapped around a user's finger F. In FIGS. 1-3, the pulse oximeter 10 can be wrapped around the side of the finger F, and in FIGS. 4 and 5, the pulse oximeter 10 can be wrapped around the tip of the finger F. In FIGS. 1-5, the pulse oximeters 10 include a multilayer substrate that is C-shaped to define a digit attacher so that the pulse oximeters 10 can be attached to a user's finger F. Instead of being wrapped around the user's finger F, it is also possible that the monitoring device is wrapped around the user's toe.

A data representation 15 can be included on the contact side of the reinforcement 80 underneath the inner transparent layer 40. Laser printing, inkjet printing and the like can be used to print the data representation on the reinforcement 80. Since the printing surface itself is covered with the inner transparent layer 40, there is no need for biocompatibility and no concern for durability against surface cleaning. Further, as shown in FIG. 5, printing can be performed more easily by providing a flat surface.

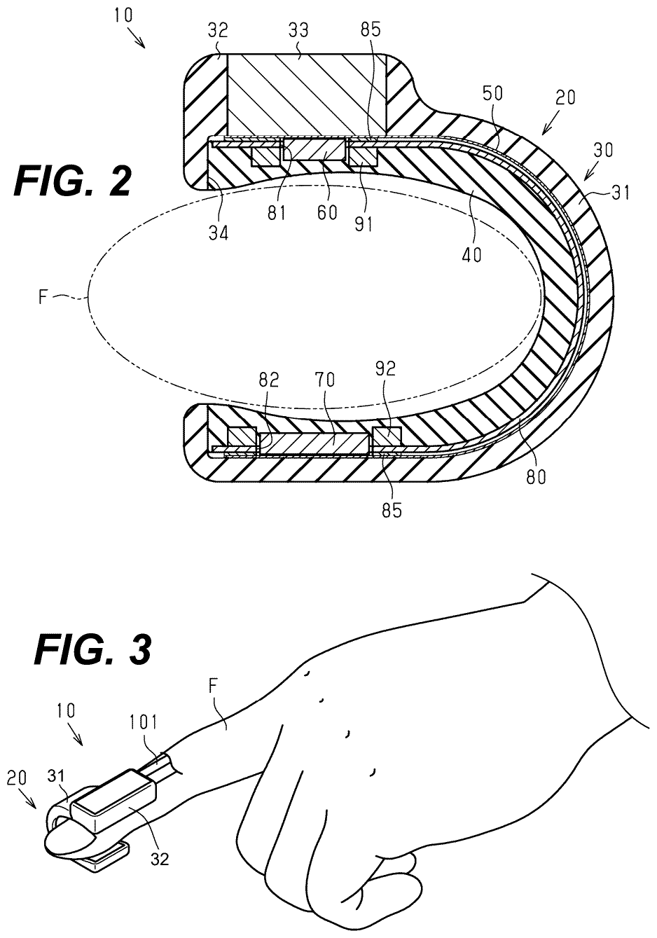

As shown in FIG. 1, a pulse oximeter 10 includes a holder 20 that can have a curved band plate form so that its two longitudinal ends are located near each other. The holder 20 can include a substrate that includes multiple layers, e.g., an inner transparent layer 40, a reinforcement 80, an adhesive layer 85, a flexible substrate 50, and an outer protective layer 31. The two ends of the holder 20 are separated and do not have to be connected to each other. More specifically, as shown in FIG. 2, the holder 20 can have a substantially C-shape in a cross-sectional view and can be curved by approximately 180° so that the two longitudinal ends are directed in the same or substantially the same direction so that the holder 20 can be attached to a digit. This arrangement of the holder 20 allows the holder 20 to define a digit attacher that can be used to attach the pulse oximeter 10 to a user's digit, e.g., a user's finger or toe.

The outer portion of the curved holder 20 includes an outer portion 30. The material of the outer portion 30 can include silicone rubber that can have a Shore A hardness of 70. It is possible to use other materials and/or other hardnesses. The Shore A hardness is obtained with a durometer that presses a cylindrical scale against the surface of the measured material to indent the material and measure the hardness from the depth of the indentation, which is the pressed depth of the scale. The color of the outer portion 30 under visible light can be black. Further, the outer portion 30 can absorb infrared light and can limit the transmission of infrared light.

As shown in FIG. 1, the outer portion 30 includes an outer protective layer 31, which can have the form of a curved band plate, and a retainer 32, which can be connected to one longitudinal end of the outer protective layer 31. When a direction that is orthogonal to the longitudinal direction of the outer protective layer 31 is defined as the widthwise direction, the retainer 32 can have a greater dimension in the widthwise direction than the outer protective layer 31. Further, the retainer 32 can have a greater thickness than the outer protective layer 31. As shown in FIG. 2, the retainer 32 includes an inner surface that is smoothly continuous with the inner surface of the outer protective layer 31 so that a step is not formed between the inner surfaces. Accordingly, a portion of the retainer 32 projects outward from the outer surface of the outer protective layer 31. The outer surface of the retainer 32 can be hollowed into a retaining cavity 33. The retaining cavity 33 can be box-shaped. The retaining cavity 33 can be hollow over substantially the entire retainer 32.

The curved inner surface of the outer portion 30 can be hollowed into a recess 34. The recess 34 can extend over substantially the entire outer portion 30 in the longitudinal direction of the outer portion 30 from the retainer 32 to the outer protective layer 31. The recess 34 can have one longitudinal end connected to the retaining cavity 33 of the retainer 32 in the thickness-wise direction of the outer portion 30. As shown in FIG. 1, the recess 34 does not have to extend to the two longitudinal ends of the outer portion 30. The recess 34 can have a dimension in the widthwise direction that is not constant over its entire region in the longitudinal direction.

As shown in FIG. 2, a flexible substrate 50 can be coupled to a bottom surface of the recess 34, that is, the curved inner surface of the outer portion 30. The flexible substrate 50 can have the form of a band plate. The flexible substrate 50 can extend over substantially the entire outer portion 30 in the longitudinal direction of the outer portion 30. The flexible substrate 50 can partition the recess 34 and the retaining cavity 33 at one longitudinal end of the recess 34. The flexible substrate 50 can have a thickness that is much smaller than that of the outer portion 30. The flexible substrate 50 can have a multilayer structure in which wiring layers are stacked with electrical insulating layers located in between. The flexible substrate 50 can have a lower flexural rigidity than the holder 20. Flexural rigidity is the resistance of a member to bending and is determined by the product of the second moment of area of the member and the Young's modulus of the material of the member.

A light emitter 60, which emits light, can be coupled to the curved inner surface of the flexible substrate 50. The light emitter 60 can be disposed on one longitudinal end of the flexible substrate 50. The light emitter 60 can be electrically connected to a wiring layer in the flexible substrate 50. The light emitter 60 can emit light inwardly from the curved holder 20. Further, the light emitter 60 can emit red light and infrared light.

As shown in FIG. 2, a light receiver 70, which receives the light emitted from the light emitter 60, can be coupled to the curved inner surface of the flexible substrate 50. The light receiver 70 can be disposed on the other longitudinal end of the flexible substrate 50 at a position opposing the light emitter 60. The light receiver 70 can be disposed along the axis of the light emitted from the light emitter 60. The light receiver 70 can be electrically connected to the wiring layer in the flexible substrate 50.

As shown in FIG. 1, a cable group 101 can extend into the retaining cavity 33 of the outer portion 30. Each cable of the cable group 101 can be connected to the flexible substrate 50 at a location in the retaining cavity 33 where the flexible substrate 50 is exposed. The light emitter 60 and the light receiver 70 can receive electric power, can transmit signals, and can send signals through the cable group 101 and the flexible substrate 50. The retaining cavity 33 can be filled with an electrical insulating resin such as a silicone potting resin or an epoxy resin. As schematically shown in FIG. 1, the other end of the cable group 101 can be connected to a power supply 102 and a measurement device 103. The power supply 102 and the measurement device 103 can be in a device not attached to the patient or can be located in a device attached to the patient (e.g., a patient-worn sensor or chest device).

As shown in FIG. 2, a reinforcement 80 can be coupled to the curved inner surface of the flexible substrate 50. The reinforcement 80 can have the form of a band plate. The reinforcement 80 can be curved to extend in conformance with the curved form of the flexible substrate 50. As shown in FIG. 2, the reinforcement 80 can function as a leaf spring. The reinforcement 80 can have substantially the same thickness as the flexible substrate 50 but can have a thickness different from the flexible substrate 50. The reinforcement 80 can have a dimension in the direction of its short side satisfying the required rigidity that it was designed to obtain. The dimension of the reinforcement 80 in the direction of its short side can be substantially the same as that of the flexible substrate 50. The dimension of the reinforcement 80 in the longitudinal direction can be substantially the same as that of the flexible substrate 50. The reinforcement 80 can include an emitter receptacle 81 at a location opposed to the light emitter 60. The light emitter 60 can be disposed inside the emitter receptacle 81. The light emitter 60 can project from the emitter receptacle 81 into the curved reinforcement 80. Further, the reinforcement 80 can include a receiver receptacle 82 at a location opposed to the light receiver 70. The light receiver 70 can be disposed inside the receiver receptacle 82. The light receiver 70 can project from the receiver receptacle 82 into the curved reinforcement 80. The material of the reinforcement 80 can include stainless steel and/or other suitable materials.

The reinforcement 80 can be adhered by an adhesive layer 85 to the flexible substrate 50. The adhesive layer 85 on the curved inner surface of the flexible substrate 50 can extend around the light emitter 60 so as to surround the light emitter 60. Further, the adhesive layer 85 on the curved inner surface of the flexible substrate 50 can extend around the light receiver 70 so as to surround the light receiver 70. The flexible substrate 50 can be fixed by the adhesive layer 85 to the reinforcement 80.

The data representation 15 can be included on the reinforcement 80 so that the data representation 15 is visible through the inner transparent layer 40. The data representation 15 can be printed on the reinforcement 80. As shown in FIG. 1, the data representation 15 can be located underneath the retainer 32. The reinforcement 80 can provide a flat surface on which the data representation 15 can be printed. The data representation 15 can be formed, either printed or otherwise, on the reinforcement 80 before or after the reinforcement 80 is bent into a C-shape. It is also possible that the substrate of the holder 20 includes a different arrangement or different number of layers, in which case the data representation 15 can be located on the layer adjacent to the inner transparent layer 40 so that the data representation can be visible through the inner transparent layer 40. The data representation 15 can include a Unique Device Identifier (UDI) that uniquely identifies the pulse oximeter 10. The data representation 15 can include a machine-readable barcode (e.g., a one-dimensional barcode and/or a two-dimensional barcode) and/or can include human-readable information (e.g., text and/or alphanumeric characters).

An emitter light shield wall 91 can project from the curved inner surface of the reinforcement 80. The emitter light shield wall 91 can extend along the edge of the emitter receptacle 81. The emitter light shield wall 91 can extend along the entire edge of the emitter receptacle 81. The emitter light shield wall 91 can include a distal end located further inward from the end of the light emitter 60 located toward the inner side of the curved flexible substrate 50. As a result, the emitter light shield wall 91 can surround the outer side of the light emitter 60 in a view of the light emitter 60 taken from the light receiver 70.

A receiver light shield wall 92 can project from the curved inner surface of the reinforcement 80. The receiver light shield wall 92 can extend along the edge of the receiver receptacle 82. The receiver light shield wall 92 can extend along the entire edge of the receiver receptacle 82. The receiver light shield wall 92 can include a distal end located further inward from the end of the light receiver 70 located toward the inner side of the curved flexible substrate 50. As a result, the receiver light shield wall 92 can surround the outer side of the light receiver 70 in a view of the light receiver 70 taken from the light emitter 60.

An inner transparent layer 40 can be fitted into the recess 34 of the outer portion 30. More specifically, the inner portion of the curved holder 20 is formed by the inner transparent layer 40. The inner transparent layer 40 can have the form of a band plate and can be shaped in correspondence with the recess 34. The inner transparent layer 40 can cover the reinforcement 80 in addition to the light emitter 60, the light receiver 70, the emitter light shield wall 91, and the receiver light shield wall 92 from the inner side in the curved direction. Thus, the reinforcement 80 can be located between the outer portion 30 and the inner transparent layer 40. That is, the reinforcement 80 can be arranged inside the holder 20.

From the light emitter 60 to the light receiver 70 in the longitudinal direction of the holder 20, the portion of the holder 20 including the outer protective layer 31 can have a constant thickness. From the light emitter 60 to the light receiver 70 in the longitudinal direction of the holder 20, the portion of the holder 20 including the outer protective layer 31 can have a substantially constant width or can have a constant width Wd. Thus, from the light emitter 60 to the light receiver 70 in the longitudinal direction of the holder 20, the portion including the outer protective layer 31 is where the cross-sectional area is the smallest in a direction orthogonal to the longitudinal direction of the holder 20.

The material of the inner transparent layer 40 can include a silicone rubber that can have a Shore A hardness of 30. It is possible to use other materials and/or other hardnesses. The inner transparent layer 40 can be formed from a softer material than the outer portion 30. The inner transparent layer 40 can be colorless and can be transparent under visible light. Further, the inner transparent layer 40 can allow transmission of a certain amount of infrared light. Accordingly, the inner transparent layer 40 can have a higher transmittance than the outer portion 30 with respect to the light emitted from the light emitter 60.

As described above, the material of the reinforcement 80 can include stainless steel. Thus, the material of the reinforcement 80 can have a Young's modulus that is greater than the Young's modulus of the material of the outer portion 30 and the Young's modulus of the material of the inner transparent layer 40. It is also possible to use a material other than stainless steel that has a Young's modulus that is greater than the Young's modulus of the material of the outer portion 30 and the Young's modulus of the material of the inner transparent layer 40.

The operation of the pulse oximeter 10 will now be described. As shown in FIG. 3, the pulse oximeter 10 can be attached to a finger F of a hand of a user when used. As explained above but not shown in FIG. 3, the pulse oximeter 10 could be attached to a toe of a foot of a user. When attaching the pulse oximeter 10 to the finger F of the user, the two longitudinal ends of the holder 20 can be separated to widen the gap between the two longitudinal ends of the holder 20. Then, the finger F is inserted into the gap between the two longitudinal ends of the holder 20. After inserting the finger F into the curved holder 20, the gap between the two longitudinal ends of the holder 20 is no longer widened. When the gap between the two longitudinal ends of the holder 20 is no longer widened, the reinforcement 80 acts to return to its curved form prior to the widening and narrow the gap between the two longitudinal ends of the holder 20. Consequently, the curved inner surface of the holder 20 can come into close contact with the finger F of the hand of the user. The double-dashed lines in FIG. 2 show the finger F.

Blood oxygen saturation level is a value indicating the percentage of hemoglobin that is bonded with oxygen in the red blood cells flowing through the artery. When hemoglobin is bonded with a large amount of oxygen, hemoglobin has a red color under visible light. When hemoglobin is not bonded with a large amount of oxygen, hemoglobin has a blackish color under visible light.

Thus, when a large amount of oxygen is bonded with hemoglobin, in the light emitted from the light emitter 60, the percentage of red light that is transmitted is large. This increases the amount of red light received by the light receiver 70. In contrast, when a large amount of oxygen is not bonded with hemoglobin, in the light emitted from the light emitter 60, the percentage of red light that is transmitted is small. This decreases the amount of red light received by the light receiver 70. In the light emitted from the light emitter 60, the percentage of infrared light that is transmitted through the finger F is constant regardless of the amount of oxygen bonded with hemoglobin. Thus, the amount of infrared light received by the light receiver 70 is not changed by the amount of oxygen bonded with hemoglobin.

The measurement device 103, which is connected to the pulse oximeter 10, calculates the blood oxygen saturation level from the ratio of the amount of infrared light received by the light receiver 70 and the amount of red light received by the light receiver 70.

FIGS. 4 and 5 show a pulse oximeter 10 that is similar to the pulse oximeter 10 of FIGS. 1-3 discussed above, but instead of being wrapped around the side of the user's finger F as shown in FIGS. 1-3, the pulse oximeter 10 of FIGS. 4 and 5 is wrapped around the tip of the user's finger F. As with the pulse oximeter 10 of FIGS. 1-3, the pulse oximeter 10 of FIGS. 4 and 5 can be wrapped around the user's toe.

In a known pulse oximeter, the data representation is located on an exterior surface of the outer protective layer. The data representation must be on the exterior surface of the outer protective layer because the outer protective layer is black. Because the data representation is on the exterior surface of the known pulse oximeter, the data representation must include a biocompatible material because the data representation can come into contact with the user and can be damaged when the pulse oximeter is cleaned.

As shown in FIG. 5, the data representation 15 can be included under the inner transparent layer 40 according to example embodiments of the present invention. The data representation 15 can include a Unique Device Identifier (UDI) that uniquely identifies the pulse oximeter 10. The data representation 15 can include a machine-readable barcode (e.g., a one-dimensional barcode and/or a two-dimensional barcode) and/or can include human-readable information (e.g., text and/or alphanumeric characters). The pulse oximeter 10 in FIGS. 4 and 5 can include a holder 20 that includes substrate with multiple layers. As with the holder 20 in FIGS. 1-3, the holder 20 in FIGS. 4 and 5, the substrate can include an inner transparent layer 40, a reinforcement 80, an adhesive layer 85, a flexible substrate 50, and an outer protective layer 31, but it is also possible to have a different arrangement and/or a different number of layers. The pulse oximeter 10 can include a cable group 101 that is attached to a power supply 102 and a measurement device 103.

In the pulse oximeters 10 described above, the material of the reinforcement 80 can have a Young's modulus that is greater than the Young's modulus of the material of each of the outer portion 30 and the inner transparent layer 40 that form the holder 20. Thus, even when a soft material such as silicone rubber is used as the material of the holder 20, the reinforcement 80 can act to appropriately increase the flexural rigidity of the pulse oximeter 10 in its entirety. Accordingly, when the holder 20 is attached to the finger F, the holder 20 is easily kept held on the finger F.

The flexible substrate 50 can be located between the outer portion 30 and the inner transparent layer 40. That is, the flexible substrate 50 can be arranged inside the holder 20. The flexible substrate 50 can have a lower flexural rigidity than the holder 20. Thus, when the pulse oximeter 10 is attached to the user's finger F, the flexible substrate 50 can be bent in conformance with the holder 20. The flexible substrate 50 can have little effect on the deformation of the holder 20.

The flexible substrate 50 can be fixed to the reinforcement 80. Further, the reinforcement 80 can have a higher flexural rigidity than the holder 20. Accordingly, the flexible substrate 50 can be fixed with greater stability than when the flexible substrate 50 is directly fixed to the holder 20.

The inner transparent layer 40 can have a lower Shore hardness A than the outer portion 30. Thus, while a certain level of rigidity is obtained for the holder 20 in its entirety, a soft material can be used as the material of the inner transparent layer 40, which is the portion that comes into close contact with the user's finger F. Further, the use of the soft inner transparent layer 40 can allow the inner transparent layer 40 to come into close contact with the inner transparent layer 40 and can limit the effect of outside light on the light receiver 70. This can improve the measurement accuracy of the pulse oximeter 10.

The light emitter 60 and the light receiver 70 can be located between the outer protective layer 31 and the inner transparent layer 40. More specifically, the light emitter 60 and the light receiver 70 are located toward the inner transparent layer 40 from the inner surface of the outer protective layer 31 and also toward the outer protective layer 31 from the outer surface of the inner transparent layer 40. Thus, light needs to be transmitted through the inner transparent layer 40 for the light receiver 70 to receive the light emitted from the light emitter 60. The transmittance of the inner transparent layer 40 with respect to the wavelength band of the light emitted from the light emitter 60 can be higher than the transmittance of the outer protective layer 31 with respect to wavelength band of the light emitted from the light emitter 60. Thus, the light emitted from the light emitter 60 can be transmitted more easily through the inner transparent layer 40 than the outer protective layer 31. This allows the light emitted from the light emitter 60 to reach the light receiver 70. Further, the transmission of the light emitted from light emitter 60 is more limited in the outer protective layer 31 than the inner transparent layer 40. Thus, natural light or the like from outside the holder 20 is also prevented from reaching the light receiver 70.

The light emitter 60 can be surrounded by the emitter light shield wall 91. Further, the light receiver 70 can be surrounded by the receiver light shield wall 92. The light shield walls 91, 92 can block the light from the light emitter 60 that is diffused in the inner transparent layer 40 and then directed toward the light receiver 70 and does not block the light from the light emitter 60 that straightly enters the light receiver 70.

The light receiver 70 can be surrounded by the receiver light shield wall 92. Thus, even when ambient light, such as light from outside the outer protective layer 31, is directed toward the light receiver 70, the receiver light shield wall 92 can block such ambient light. This can prevent the light receiver 70 from receiving such ambient light.

In the pulse oximeters 10 in FIGS. 1-5, the holder 20 can have any shape as long as it is curved to define a digit attacher that can clamp the finger F or toe of a user. For example, the holder 20 can be shaped to have the form of a curved rod.

The holder 20 can be curved by any amount. For example, the holder 20 can extend to be curved by 360° or greater. When curved by 360° or greater, the digit can be fitted inside the holder 20 by separating the two ends of the holder 20 to form a gap.

A wireless communication device may be incorporated inside the pulse oximeters 10, for example, in the retainer 32 of the outer portion 30, to send and receive signals through wireless communication. The measurement device 103, which can be connected by wireless communication to the pulse oximeter 10, can be shared by a plurality of pulse oximeters 10. In other words, a plurality of pulse oximeters 10 can be managed by a single measurement device 103.

In the pulse oximeters 10 in FIGS. 1-5, the reinforcement 80 only needs to have a curved form and is not limited to the form of a band plate. For example, the reinforcement 80 may be shaped to have the form of a curved rod.

A plurality of reinforcements 80 can be disposed inside the holder 20. As long as the reinforcement 80 is plate-shaped, an appropriately high flexural rigidity can be obtained for the holder 20 by enlarging the cross-sectional area without overly increasing the thickness of the holder 20. If a plurality of reinforcements 80 is used, then the data representation can be included on the reinforcement 80 adjacent to the inner transparent layer 40.

The material of the reinforcement 80 is not limited to the materials described above. The material of the reinforcement 80 only needs to have a Young's modulus that is greater than the Young's modulus of the holder 20. For example, the material of the reinforcement 80 can be a resin having a greater Young's modulus than the material of the holder 20 or may be a metal such as aluminum.

The material of the outer portion 30, including the outer protective layer 31, is not limited to the materials discussed above. For example, the material of the outer portion 30, including the outer protective layer 31, can be a material of which the Shore A hardness is lower than or equal to that of the inner transparent layer 40. Preferably, the outer protective layer 31 is formed from rubber because when the pulse oximeter 10 is attached to the user's digit, the outer protective layer 31 comes into contact with the user or a person who is attaching the pulse oximeter 10 to the user. The material of at least the outer portion 30, including the outer protective layer 31, only needs to have a Young's modulus that is smaller than the Young's modulus of the reinforcement 80.

The hardness of the outer protective layer 31 and the hardness of the inner transparent layer 40 are not limited to Shore A hardness. For example, Shore D hardness or Vickers hardness may be used, and at least only the inner transparent layer 40 needs to be softer than the outer protective layer 31.

The material of the inner transparent layer 40 is not limited to the materials discussed above. For example, the material of the inner transparent layer 40 can be a material of which the Shore A hardness is higher than or equal to that of the outer protective layer 31. Preferably, the inner transparent layer 40 is formed from rubber because the inner transparent layer 40 directly comes into contact with the user. The material of at least the inner transparent layer 40 can have a Young's modulus that is lower than the Young's modulus of the reinforcement 80.

The material of the inner transparent layer 40 does not need to have a lower flexural rigidity than the holder 20 in its entirety. For example, the flexural rigidity of the flexible substrate 50 between the light emitter 60 and the light receiver 70 in the longitudinal direction of the holder 20 can be lower at the portion including the outer protective layer 31 than the portion where the cross-sectional area is the smallest in the direction orthogonal to the longitudinal direction of the holder 20. Further, the flexural rigidity of the flexible substrate 50 can be higher than the flexural rigidity of the holder 20. Even in this case, the arrangement of the reinforcement 80 inside the holder 20 returns the deformed reinforcement 80 to its original form. This can improve the holding force of the holder 20.

The flexible substrate 50 can be omitted. For example, the light receiver 70 can be connected to a cable separate from the cable group 101, and the separate cable may be connected to the measurement device 103. In this case, because there is no need for cables to be bundled into the cable group 101, the flexible substrate 50 can be omitted.

The transmittance of the outer protective layer 31 and the transmittance of the inner transparent layer 40 are not limited to the example discussed above. For example, as long as the inner transparent layer 40 includes holes in the thickness-wise direction of the holder 20 at positions aligned with the light emitter 60 and the light receiver 70, the light emitted from the light emitter 60 can pass through the corresponding hole and be transmitted through the user's digit. Further, the light transmitted through the user's digit can pass through the hole at the side of the light receiver 70 and be received by the light receiver 70. Thus, for example, the inner transparent layer 40 does not have to transmit the light emitted from the light emitter 60. The portion of the inner transparent layer 40 covering the data representation should have a transmittance that allows the data representation to be visible through the inner transparent layer 40.

The inner transparent layer 40 does not have to be entirely colorless and transparent. The inner transparent layer 40 may be colorless and transparent at only locations corresponding to the light emitter 60, the light receiver 70, and the data representation 15, and the other portions may be colored in black under visible light. In this case, the light emitted from the light emitter 60 is transmitted through the inner transparent layer 40 and received by the light receiver 70 in the same manner as when there are holes as described above.

The emitter light shield wall 91 does not need to be shaped as a quadrangular frame like in the above example embodiment. The emitter light shield wall 91 can be disposed in the range where light has to be blocked. For example, a portion at a first longitudinal end side of the holder 20 may be omitted. The same applies to the receiver light shield wall 92.

The emitter light shield wall 91 can be omitted. For example, if the light emitter 60 has high directivity, then the emitter light shield wall 91 can be omitted if the inner transparent layer 40 impedes the transmission of light from the light emitter 60 that does not pass through the user's digit.

The receiver light shield wall 92 can be omitted. For example, if the light receiver 70 has high directivity, then the receiver light shield wall 92 can be omitted if the light receiver 70 does not receive the light that is not transmitted through the user's digit but is transmitted through the inner transparent layer 40.

The pulse oximeters 10 can measure the pulse instead of or in addition to the blood oxygen saturation level. When comparing the amount of red light and infrared light received by the light receiver 70 as described above, fluctuations in the amount of light received by the light receiver 70 can be measured to measure the pulse.

As explained above, the holder 20 can wrap around the side of the finger F or around the tip of the finger F.

The set of the light emitter 60 and the light receiver 70 can be referred to as an optical physiological sensor including a light emitting element and a light receiving element. The holder 20 of can be referred to as a C-shaped elastic probe or hingeless elastic probe that accommodates the light emitter 60 and the light receiver 70 and can be configured to be elastically attached to the periphery (e.g., finger F or toe) of a measured subject to position the light emitter 60 and the light receiver 70 at different locations on the surface (skin) of the periphery of the measured subject. The outer protective layer 31 and the inner transparent layer 40 can be collectively be referred to as the skin layer, and the outer protective layer 31 and the inner transparent layer 40 can respectively be referred to as the outer skin layer and the inner skin layer. One or both of the outer protective layer 31 and the inner transparent layer 40 can be partially or entirely formed from an electrically insulating cushion material. The flexible substrate 50 can be referred to as a circuit substrate that is electrically connected to the optical physiological sensors (e.g., light emitter 60, light receiver 70). The reinforcement 80 can be referred to as a curved core layer or a curved plate reinforcement core including a length, a thickness, a first position (emitter receptacle 81) corresponding to the light emitter 60, a second position (receiver receptacle 82) corresponding to the light receiver 70 and separated from the first position in the longitudinal direction of the reinforcement 80, and a curved middle portion located between the first position and the second position.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A monitoring device comprising:
a transparent layer; and
a data representation located underneath the transparent layer such that the data representation is visible through the transparent layer; wherein
the monitoring device is configured to monitor a user when the monitoring device is attached to the user.

2. The monitoring device of claim 1, wherein the data representation includes a Unique Device Identifier of the monitoring device.

3. The monitoring device of claim 1, wherein the data representation includes a machine-readable barcode.

4. The monitoring device of claim 3, wherein the machine-readable barcode includes a one-dimensional barcode and/or a two-dimensional barcode.

5. The monitoring device of claim 1, wherein the data representation includes human-readable information.

6. The monitoring device of claim 5, wherein the human-readable information is text and/or alphanumeric characters.

7. The monitoring device of claim 1, wherein the data representation includes a printed data representation.

8. The monitoring device of claim 1, wherein the transparent layer includes rubber.

9. The monitoring device of claim 1, wherein
the monitoring device has a C-shape; and
the transparent layer is on an inner surface of the monitoring device.

10. The monitoring device of claim 1, wherein the monitoring device is configured such that the transparent layer is in direct contact with the user when the monitoring device is attached to the user.

11. The monitoring device of claim 1, wherein the monitoring device includes a digit attacher configured to be attached to a finger of the user such that the transparent layer is in direct contact with the finger of the user when the monitoring device is attached to the finger of the user.

12. A monitoring device system comprising the monitoring device of claim 1 attached to the user.

13. The monitoring device system of claim 12, wherein the monitoring device is attached to a finger of the user.

14. The monitoring device system of claim 13, wherein the transparent layer is in direct contact with the finger of the user.

15. The monitoring device system of claim 12, further comprising:
a power supply connected to the monitoring device; and
a measurement device connected to the monitoring device.

16. A monitoring device:
a substrate having a C-shape and configured so that the monitoring device can be attached to a user's digit;
a transparent layer that is on an inner surface of the substrate and that directly contacts the user's digit when the monitoring device is attached to the user's digit; and
a data representation located under the transparent layer such that the data representation can be viewed through the transparent layer.

17. The monitoring device of claim 16, wherein the data representation includes a Unique Device Identifier.

18. The monitoring device of claim 16, wherein the data representation includes a machine-readable barcode.

19. The monitoring device of claim 16, wherein the data representation includes human-readable information.

20. The monitoring device of claim 16, wherein the transparent layer includes rubber.
